(19) United States
(12) Patent Application Publication
(10) Pub. No.: US 2019/0261959 A1
(43) Pub. Date: Aug. 29, 2019

(54) REAL-TIME TELE-SONOGRAPHY

(71) Applicant: Cedars-Sinai Medical Center, Los Angeles, CA (US)

(72) Inventor: Jonathan Frankel, Los Angeles, CA (US)

(73) Assignee: Cedars-Sinai Medical Center, Los Angeles, CA (US)

(21) Appl. No.: 16/346,767

(22) PCT Filed: Nov. 21, 2017
(86) PCT No.: PCT/US2017/062880

§ 371 Date: May 1, 2019

Related U.S. Application Data
(60) Provisional application No. 62/425,413, filed on Nov. 22, 2016.

(51) Int. Cl. A61B 8/00; B25J 3/04; G09B 23/28; G16H 40/67

(52) U.S. Cl. CPC A61B 8/565; A61B 8/587; A61B 8/4218; A61B 8/4263; G16H 40/67; A61B 8/54; B25J 3/04; G09B 23/286; A61B 8/429

(57) ABSTRACT

Aspects of the present disclosure relate to a master sonography device configured to determine movement parameters describing movement of a master transducer head by a sonographer, and a slave sonography device, remote from the master sonography device, being configured to move a slave transducer head based on the movement parameters received from the master sonography device. The slave sonography device includes a positioning system that is configured to generate patient parameters describing margins of a patient. Aspects of the present disclosure further relate to a mock patient configured to conform to the margins of the patient based on the patient parameters to mimic the patient at the master sonography device during the sonography examination, such that manipulation of the master transducer head applied against the mock patient controls movement of the slave transducer head applied against the patient.

### CROSS-REFERENCE TO RELATED APPLICATIONS

The present application claims the benefit of and priority to U.S. Provisional Application No. 62/425,413, filed Nov. 22, 2016, and entitled, “REAL-TIME TELE-SONOGRAPHY,” which is hereby incorporated by reference herein in its entirety.

### FIELD

The present invention relates to sonography devices, systems, and methods used in medical examinations and/or procedures. More particularly, the present invention relates to removing the requirement of having the sonographer at the patient during a sonography examination.

### BACKGROUND

The value of diagnostic quality sonography for the general medical community is incontrovertible. Its use is nearly ubiquitous, spanning almost every medical specialty. Whether used in the emergent setting for diagnosing surgical abdomens or the outpatient setting for evaluation of renal stones and/or liver disease, the use of sonography as an affordable, safe, and convenient means of obtaining medical imaging critical to guiding patient management is evident and only increasing.

The greatest hurdle to prolific use of sonography is the expense of the equipment required to perform sonography, the expertise required for the equipment's operation, and the medical training to interpret the information obtained. Even if sonography equipment were inexpensive and universally accessible, sonography's application would still be limited by the availability of qualified sonographers to reliably provide images of diagnostic quality. Furthermore, the expense of a certified radiologist to interpret each set of images further financially limits sonography's use.

Upwards of 65% of medical facilities have limited this expense by removing the radiologist from the equation and rely on the skill of the sonographer to provide images of enough quality that the referring specialist (e.g., obstetrics, urology, emergency medicine, cardiology, etc.) can confidently make the diagnosis themselves without the need for an expert opinion. While this has served some facilities to reduce cost, there are still more private practices that require further financial incentives to make wider use of sonography. Moreover, the general internist or family medicine practitioner lacks the time and breadth of knowledge to reliably diagnose the variety of pathology obtainable from routine sonography examinations performed on their diverse patient population. These offices are left with the option of referring their sonography needs to third parties that possess the equipment, the sonographers, and the radiologists to provide a convenient service for a fee. While this arrangement serves to reduce the cost for the referrer, it nonetheless results in a significant expense, especially relative to the cost of a single sonography machine and the salary of a sonographer. The average diagnostic quality sonography machine will range from $25,000 to $75,000; with most large hospitals spending upwards of $120,000. Each machine has an expected lifespan of 6-8 years, and the average sonographer makes around $89,000. Therefore, a theoretical minimal first year expense for sonographic images could be as low as $150,000, with a continuing annual expense of at least $89,000, not including insuring the machinery and cost of repairs, which are unpredictable but expensive when necessary. When confronted with these costs, it is not surprising that nearly 50% of medically indicated sonography examinations are referred to outpatient imaging centers to obtain otherwise routine sonography imaging. Referring a patient to an outside imaging facility accomplishes the imaging goal but at the cost of additional time, stress, and expense for the patient required now to visit two medical facilities for one problem.

Whether performed at a dedicated radiology center or specialty practices that possess their own equipment and sonographers, the theoretical annual cost of sonography ownership is proportional to the number of sonographers in the department. Therefore, the expense of the sonographer is highly scrutinized; attempting to ensure his or her time is efficiently utilized to justify the expense of employing fulltime or even part time sonographic coverage. Ideally, given unpredictable service volume, the additional expense of a sonographer's salary could be reduced to a minimum if the sonographer were paid only for services rendered; an arrangement not often realistic, requiring absorbing the expense of lost productivity time against the possibility of being understaffed during crucial times to ensure patient care is optimized as well as imaging income remains within the department.

The conclusion is that the limiting factor preventing universal use of sonography in all medical facilities is the cost of tangible human operators; sonographers. If the sonography machine was a singular technology purchase from which its benefit could be realized without the need for a human to be present to expertly guide its functioning, then its life-long expense could be greatly reduced and its benefit provided to even the most inaccessible locales. The expense of sonography would then only be the initial purchase price, maintenance/insurance, and the cost of each individual examination performed.

The first iteration of technology attempting to manifest a solution to the foregoing has already been implemented in the form of sonography using remote analysis of the generated images. Specifically, sonography technicians can send their images to a centrally located radiologist via telecommunication services and obtain expert medical readings of their images. This service has proved itself beneficial to hospitals with limited overnight radiologic coverage as well as distant medical practices that can only afford a sonographer and a sonography machine. However, this solution is still too expensive and technically demanding for a significant percentage of the population. The need for a properly trained sonographer to be present at the patient's bed side to perform the examination not only limits those facilities that can afford the additional expense of a sonographers fee, in addition to the sonography machine, but it also requires that the sonographer possess adequate skills to perform the various examinations that may arise and be able to provide diagnostic quality images to be interpreted by the radiologist. If the sonographer is unsure of the technique for the examination, has difficulty with the anatomy or is uncertain of the findings, communicating these issues with a radiologist via a telecommunication systems is limited at best. The result is suboptimal imaging and incomplete diagnoses.

According to the foregoing issues, a need exists that allows for remote sonography examinations that do not suffer from poor quality images and allow sonographers the same freedom as if they were at the patients' bedsides.

### SUMMARY

An aspect of the present disclosure includes a tele-sonography system. The system includes a master sonography device that includes a master transducer head. The master sonography device is configured to determine movement parameters describing movement of the master transducer head by a sonographer at the master sonography device. The system also includes a slave sonography device, remote from the master sonography device that includes a slave transducer head and a positioning system. The slave sonography device is configured to move the slave transducer head based on the movement parameters received from the master sonography device. The positioning system is configured to generate patient parameters describing margins of a patient upon which a sonography examination is performed at the slave sonography device. The system also includes a mock patient, at the master sonography device, configured to conform to the margins of the patient based on the patient parameters and mimic the patient at the master sonography device during the sonography examination. Manipulation of the master transducer head applied against the mock patient controls movement of the slave transducer head applied against the patient.

Another aspect of the present disclosure includes a method of performing tele-sonography. The method includes positioning a patient relative to a slave sonography device, with the slave sonography device including a slave transducer arm, a slave transducer head, and a positioning system. The slave sonography device is configured to move the slave transducer head based on movement parameters received over a communications network. The method further includes acquiring patient parameters describing margins of the patient relative to the slave sonography device, and generating a model of the patient based on the patient parameters. The method further includes applying the model to a mock patient, adjacent a master sonography device and remote from the slave sonography device, to conform margins of the mock patient to the margins of the patient, and acquiring sonography images at the slave sonography device based on control of the slave sonography device by the master sonography device.

These and other capabilities of the disclosed apparatuses and methods will be more fully understood after a review of the following figures, detailed description, and claims.

### BRIEF DESCRIPTION OF THE DRAWINGS

FIG. 1 shows a schematic view of a real-time tele-sonography system, according to aspects of the present disclosure.

FIG. 2 shows a perspective view of the slave sonography site within the system of FIG. 1, in accord with aspects of the present disclosure.

FIG. 3 shows a perspective view of the master sonography site within the system of FIG. 1, in accord with aspects of the present disclosure.

FIG. 4A shows a perspective view of a mock patient in a deflated state, in accord with aspects of the present disclosure.

FIG. 4B shows a perspective view of the mock patient of FIG. 4A in a first inflated state, in accord with aspects of the present disclosure.

FIG. 4C shows a perspective view of the mock patient of FIG. 4A in a second inflated state, in accord with aspects of the present disclosure.

FIG. 5 shows a perspective view of a loading/unloading arm of a slave sonography device, in accord with aspects of the present disclosure.

FIG. 6 shows perspective view of a drum for holding additional transducer heads for a slave sonography device, in accord with aspects of the present disclosure.

FIG. 7A shows a perspective view of a transducer head with a catheter for distributing gel on the transducer head, in accord with aspects of the present disclosure.

FIG. 7B shows a perspective view of an alternative transducer head with a catheter for distributing gel on the transducer head, in accord with aspects of the present disclosure.

FIG. 7C shows a perspective view of application of gel relative to the transducer head in FIG. 7B, in accord with aspects of the present disclosure.

FIG. 7D shows a perspective view of an alternative transducer head with a catheter for distributing gel on the transducer head, in accord with aspects of the present disclosure.

FIG. 7E shows a perspective view of application of gel relative to the transducer head in FIG. 7D, in accord with aspects of the present disclosure.

FIG. 8 shows a perspective view of the slave sonography device 200, in accord with aspects of the present disclosure.

FIG. 9 is a flowchart of a procedure for tele-sonography, in accord with aspects of the present disclosure.

While the apparatuses and methods discussed herein are susceptible to various modifications and alternative forms, specific embodiments have been shown by way of example in the drawings and will be described in detail herein. It should be understood, however, that the description is not intended to be limited to the particular forms disclosed. Rather, the description is to cover all modifications, equivalents, and alternatives falling within the spirit and scope of the present disclosure as defined by the appended claims.

### DETAILED DESCRIPTION

While the apparatuses discussed in the present disclosure are susceptible of embodiment in many different forms, there is shown in the drawings and will herein be described in detail preferred embodiments of the apparatuses with the understanding that the present disclosure is to be considered as an exemplification of the principles of the apparatuses and is not intended to limit the broad aspect of the apparatuses to the embodiments illustrated. For purposes of the present detailed description, the singular includes the plural and vice versa (unless specifically disclaimed); the word “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

While there are technologies that exist that enable some degree of remote control of a transducer head, there is no technology that allows for the degree of precision and remote accuracy on a subject with both afferent and efferent information communicated simultaneously to the sonographer as well as to the patient. The technology discussed herein provides a convenient and simple modality to be readily implemented without requiring onsite training prior to use. Once the patient is positioned, the equipment is completely controlled by the sonographer remotely.

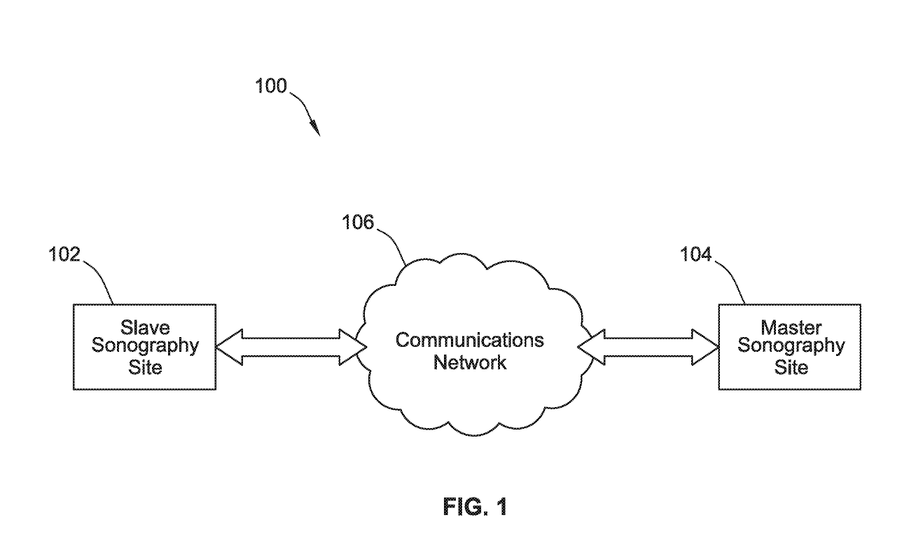

FIG. 1 shows a schematic view of a real-time tele-sonography system 100, according to aspects of the present concepts. The system 100 includes a slave sonography site 102, a master sonography site 104, and a communications network 106. As described in detail below, the slave sonography site 102 is where a patient 204 on which a sonography examination is performed is located. As described in detail below, the master sonography site 104 is where the sonographer performing the examination is located, remote from the patient 204. The communications network 106 allows for the transmission of information (e.g., sonography information) between the slave sonography site 102 and the master sonography site 104 in real time as the sonography examination is being performed.

Although FIG. 1 includes only one slave sonography site 102, the system 100 can include any number of slave sonography sites 102. For example, multiple slave sonography sites 102 can all communicate through the communications network 106 with one master sonography site 104. Similarly, although FIG. 1 includes only one master sonography site 104, the system can include any number of master sonography sites 104. For example, the slave sonography site 102 can communicate will multiple different master sonography sites 104, either concurrently for one sonography examination or consecutively for multiple sonography examinations. In some aspects, depending on the sonography examination being performed, the slave sonography site 102 can communicate with a different one of a plurality of master sonography sites 104, such as the master sonography site 104 that specializes in the particular sonography examination being performed. Further, the system 100 can have multiple slave sonography sites 102 and multiple sonography sites 104, and even multiple communications networks 106. However, for ease of explanation of the various concepts described herein, a single slave sonography site 102 and a single master sonography site 104 are referred to below.

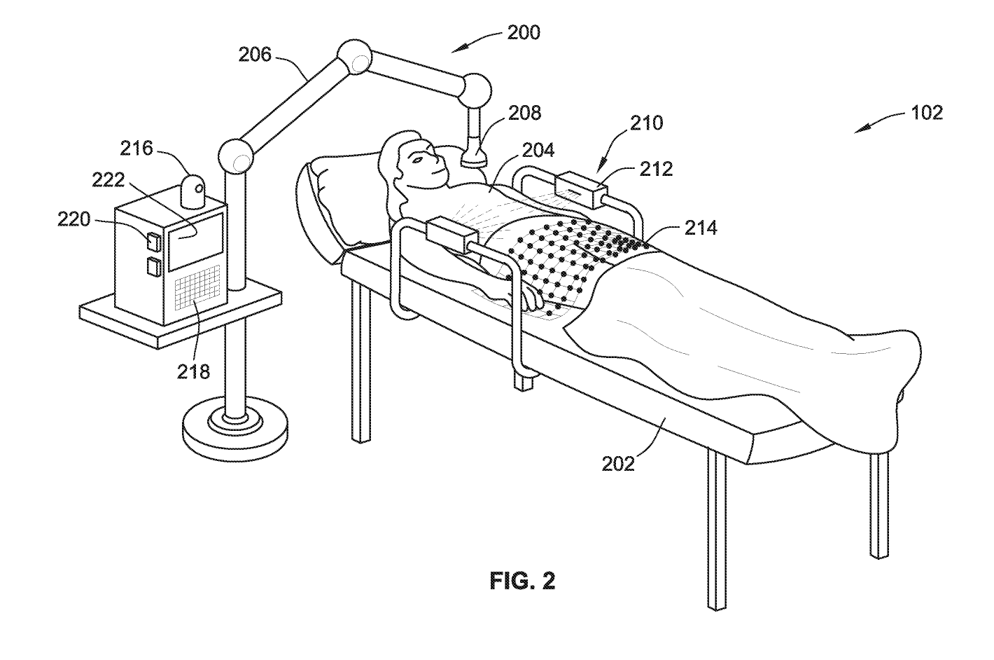

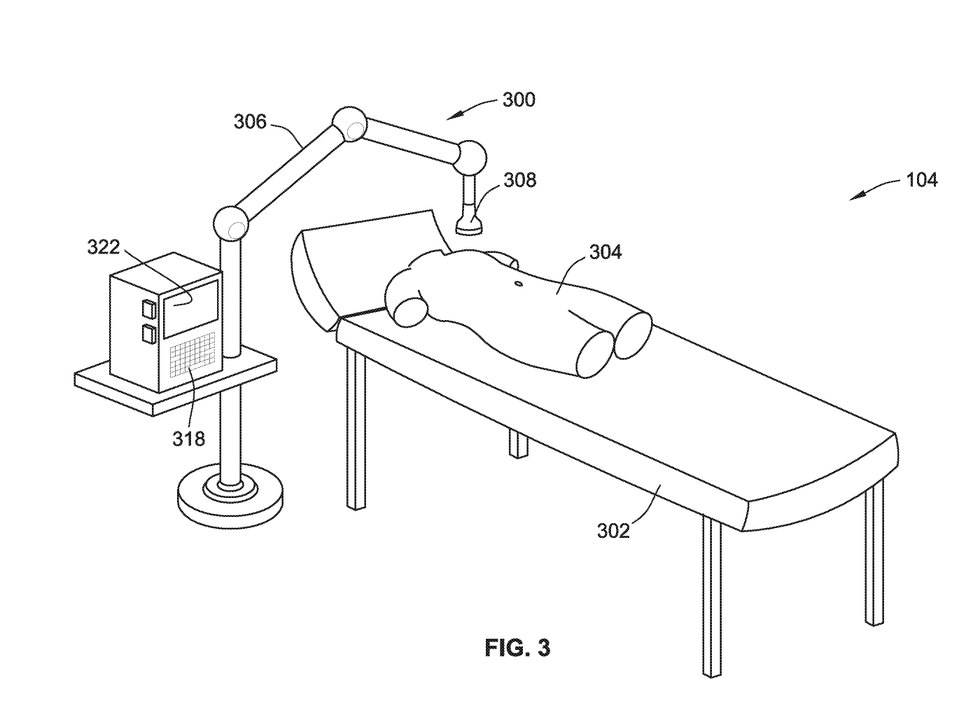

The system 100 solves the problems discussed above with conventional sonography by allowing the sonographer performing the sonography examination, as well as a radiologist that examines the images generated from the sonography examination, to be remote from the patient 204. However, the system 100 allows for accurate and precise sonography examinations despite the remote arrangement of the sonographer from the patient 204 (FIG. 2) based on the control between the slave and master sonography sites 102, 104, as well as for the mock patient 304 (FIG. 3) used at the master sonography site 104, as discussed in detail below. Accordingly, the slave sonography site 102 can be at various locations and/or facilities that otherwise could not support an entire sonography examination practice or facility. For example, the slave sonography site 102 can be any one of the locations discussed above, such as primary care and family medicine offices, subspecialty clinics, private practices, hospitals in more secluded regions, and rural and some suburban secondary care centers. The master sonography site 104 can be any facility or location that can support a sonography examination practice or facility, such as supporting the personnel required for obtaining and analyzing images generated during sonography examinations, such as full-time sonographers and radiologists. For example, the master sonography site 104 can be locations such as large hospitals in urban areas.

The communications network 106 of the system 100 can include one or more networks, such as a data network, a wireless network, a telephony network, or any combination thereof. The data network can be any local area network (LAN), metropolitan area network (MAN), wide area network (WAN), a public data network (e.g., the Internet), a short range wireless network, or any other suitable packet-switched network, such as a commercially owned, proprietary packet-switched network (e.g., a proprietary cable or fiber-optic network), and the like, or any combination thereof. The wireless network can be, for example, a cellular network that can employ various technologies, including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless mediums, worldwide interoperability for microwave access (WiMAX), Long Term Evolution (LTE) networks, code division multiple access (CDMA), wideband code division multiple access (WCDMA), wireless fidelity (WiFi), wireless LAN (WLAN), Bluetooth®, near field communication (NFC), Internet Protocol (IP) data casting, digital radio/television broadcasting, satellite, mobile ad-hoc network (MANET), and the like, or any combination thereof. Further, the slave sonography site 102 and the master sonography site 104 can communicate with each other over the communications network 106 using well known, new, or still developing protocols.

FIG. 2 shows a perspective view of the slave sonography site 102, in accord with aspects of the present disclosure. The slave sonography site 102 includes a slave sonography device 200, a gurney 202, and the patient 204 upon which the sonography examination is performed. The slave sonography device 200 includes a transducer arm 206 and a transducer head 208. The slave sonography device 200 is configured to manipulate the transducer arm 206 and the transducer head 208 to (e.g., without manipulation of the transducer arm 206 or the transducer head 208 by a sonographer or other operator at the slave sonography site 102) perform a sonography examination on the patient 204 based on information received from the master sonography site 104. In particular, once the transducer arm 206 and the transducer head 208 are positioned against or in proximity to the patient 204, as instructed (e.g., from the master sonography site 104), the slave sonography device 200 is configured to manipulate the transducer head 208 against the patient 204 based on commands received from the master sonography site 104 to perform a sonography examination.

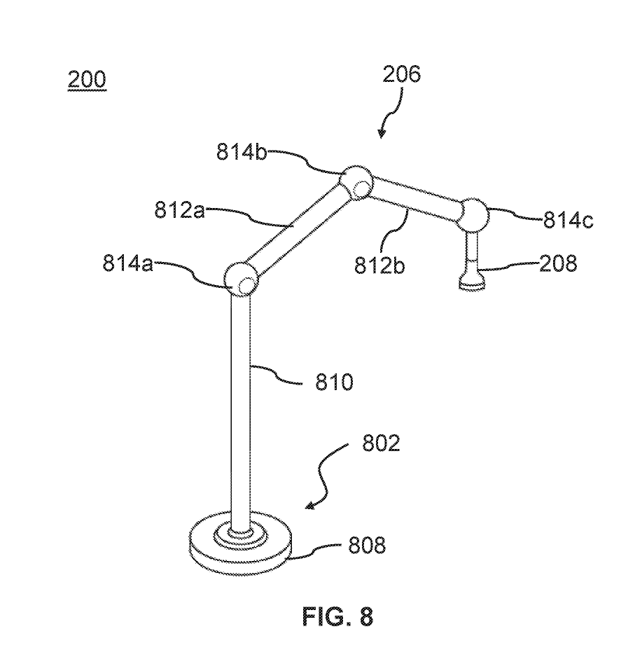

The slave sonography device 200 includes mechanisms configured to move the transducer arm 206 and the transducer head 208 and apply pressure against the patient 204 with the transducer head 208. As discussed further below in relation to FIG. 8, the slave sonography device 200 can include, for example, one or more motors, actuators, pumps, and the like to move the components of the slave sonography device 200, such as the transducer arm 206, one or more segments of the transducer arm 206, and the transducer head 208. Accordingly, movement of the slave sonography device 200 is fully autonomous upon receiving commands from the master sonography site 104. Moreover, the movement of the slave sonography device 200 mimics the movement of the device as if a sonographer were present at the slave sonography site 102, manually moving the device.

In some aspects, the slave sonography device 200 can be a free-standing device positioned adjacent the gurney 202, such as shown in FIG. 2. In such a configuration, all components of the slave sonography device 200 can be portable and can extend from the slave sonography device 200 to access the patient 204. Therefore, the slave sonography device 200 can be mobile and capable of being used in a sonography suite, an emergency room, in-patient rooms, and the like. In some aspects, the slave sonography device 200 can be coupled to and supported by the gurney 202. For example, the slave sonography device 200 can be connected to an arch or an arm, which in turn is connected to the gurney 202, such as via side rails of the gurney 202. In some aspects, the slave sonography device 200 can be mounted and/or fixed to the floor, one or more walls, and/or the ceiling of a room containing the slave sonography device 200. In some aspects, the slave sonography device 200 can be a compression assist device.

The slave sonography device 200 can be positioned relative to the patient 204 by any operator, such as a nurse, an aid, or a layperson, and does not require a sonographer at the slave sonography site 102 to position the device. After being placed relative to the patient 204, the slave sonography device 200 is configured to identify the patient 204's arrangement (e.g., orientation, location, and position) with respect to the slave sonography device 200. To identify the patient 204's arrangement, the slave sonography device 200 includes a positioning system 210. The positioning system 210 is configured to determine the arrangement of the patient 204 relative to the slave sonography device 200 so as to provide a frame of reference for how movements of the transducer arm 206 and the transducer head 208 relate to the patient 204.

In some aspects, the positioning system 210 consists of a laser imaging system 212 for identifying the patient 204's arrangement. The laser imaging system 212 can obtain the patient 204's arrangement from either one or both sides of the patient 204, the top of the patient 204, the bottom of the patient 204, or a combination thereof by detectors either on the gurney 202 (as shown in FIG. 2), arms extending from the slave sonography device 200, or a combination thereof. The operator of the slave sonography device 200 can extend the arms and/or arrange the detectors about the gurney 202 after arranging the patient 204 on the gurney 202.

Alternatively, or in addition, in some aspects the positioning system 210 consists of a plurality of tactile sensors 214 placed on the patient 204. The plurality of tactile sensors 214 can determine the margins of the patient 204 by being placed strategically on the patient 204 at the most superior, inferior, and lateral aspects of the patient 204, such as at the lower abdomen, the mid abdomen, the thorax, etc. Each tactile sensor of the plurality of tactile sensors 214 can determine its relative position among the plurality of tactile sensors 214 and the slave sonography device 200, enabling accurate coordination between the transducer arm 206, the transducer head 208, and the physical position of the patient 204.

As discussed in greater detail below, the operator at the slave sonography site 102 can direct the patient 204 to a specific arrangement on the gurney 202. Once the patient 204 is arranged on the gurney 202, the transducer arm 206 of the slave sonography device 200 can be placed into the initiation position by the operator of the slave sonography device 200, and the operator of the slave sonography device 200 can indicate completion of the arrangement of the patient 204 and the placement of the slave sonography device 200 in the initiation position. In some aspects, the slave sonography device 200 can include a confirmation button to signal to the master sonography site 104 that the slave sonography site 102 is ready for the sonography examination to begin.

In some aspects, the slave sonography device 200 can include a camera 216 to provide a video feed of the patient 204 to the master sonography device 300 during the examination. The camera 216 can be located relative to the patient 204 according to various arrangements, and the arrangements can vary according to the specific examinations being performed. Moreover, in some aspects, the camera 216 can be positioned according to the sonographer's natural position as if the sonographer was performing the examination at the patient 204 rather than remote to the patient 204. In some aspects, the camera 216 can also be controlled by the sonographer so that the view of the camera 216 can be modified as required for the sonographer during an examination. The slave sonography device 200 can further include a speaker 218 to provide audio commands at the slave sonography site 102, either from the master sonography site 104 or stored within the slave sonography device 200. The slave sonography device 200 can also include a microphone 220 so that the operator and/or the patient 204 at the slave sonography site 102 can verbally communicate with the sonographer at the master sonography site 104.

In some aspects, the slave sonography device 200 can include a maximum pressure limit that can represent the maximum pressure that the transducer arm 206 or the transducer head 208 can apply against the patient 204. The maximum pressure can be dependent on the specific examination being performed based on some examinations requiring and/or allowing for greater pressures than others. By including a maximum pressure limit, if a mistake in positioning the slave sonography device 200 or the patient 204 results in an incorrect placement of the transducer head 208 relative to the patient 204, physical injury to the patient 204 can be limited or prevented. In some aspects, the maximum pressure limit can be, for example, 1, 2, 3, 5, 10, 15, 25, or 50 pounds per square inch (lbs/in²).

The slave sonography device 200 can include a display 222 for the sonographer at the master sonography site to show one or more commands or instructions for the operator to perform at the slave sonography site 102, and other information or graphical user interface elements. In some aspects, the slave sonography device 200 can include an emergency shutoff for the operator at the slave sonography site 102 to activate at any time to release the transducer arm 206 and retract all components of the slave sonography device 200 from the patient. The emergency shutoff can be, for example, a manual button on the slave sonography device 200 or a graphical button, such as on the display of the slave sonography device 200. The emergency shutoff can override all control from the master sonography device 300 (FIG. 3) to limit or prevent injury to the patient 204. Similarly, this shutoff switch can be provided additionally to the patient to hold during the examination to immediately release pressure or pause the examination determined at the patient's discretion.

FIG. 3 shows a perspective view of the master sonography site 104, in accord with aspects of the present disclosure. The master sonography site includes the master sonography device 300, a gurney 302, and a mock patient 304. The master sonography device 300 can be similar to the slave sonography device 200 discussed above and include a transducer arm 306 and a transducer head 308. In some aspects, the master sonography device 300 can lack the mechanisms for automatically controlling the position of the transducer arm 306 and the transducer head 308, such as the motors, actuators, and pumps discussed above that allow the master sonography device 300 to move independently from manual control by a sonographer. Instead, the master sonography device 300 can include one or more sensors that translate the positioning of the master sonography device 300 relative to the mock patient 304 into instructions for positioning the slave sonography device 200 relative to the patient 204, as discussed in greater detail below.

In some aspects, the master sonography device 300 can include the mechanisms that provide independent control of the master sonography device 300, and the mechanisms can include the sensors for generating the information. In some aspects, the mechanisms on the master sonography device 300 can provide assistance, such as compression assistance, to alleviate the strain typically experienced by sonographers in positioning the transducer head 308 against a patient 204 (or the mock patient 304, in the case of the master sonography site 104) to conduct the sonography examination. In such aspects, the master sonography device 300 can be a compression assist device. In some aspects, the mechanisms on the master sonography device 300 can provide resistance, rather than assistance, or in addition to assistance, to the sonographer to reflect the amount of force or pressure generated against the patient 204 by the transducer head 208 at the slave sonography site 102. Also, the mechanisms that provide independent control of the master sonography device 300 allow for haptic and tactile feedback to be transmitted from the slave sonography device 200 allowing the sonographer controlling the master sonography device 300 to perceive in real-time the physical pressure and recoil of the transducer head 308 on the patient 204, allowing for a more natural examination, similar to a standard in-person sonographic examination.

In some aspects, the changes in position of the master sonography device 300 are mimicked by the slave sonography device 200 in a default 1-to-1 (1:1) relationship. For instance, a movement of the master sonography device 300, such as the transducer arm 306 or the transducer head 308, 10 cm to the left can result in the slave sonography device 200, such as the transducer arm 206 or the transducer head 208, mimicking the motion by performing a movement 10 cm to the left. The 1:1 relationship allows for the sonographer to interact with the patient through the interface seamlessly as if performing the examination at the patient's bedside. Alternatively, the system 100 could be modified by the sonographer to allow for even greater or reduced sensitivity. For example, the relationship between the movement of the master sonography device 300 relative to the slave sonography device 200 (master:slave) can be 1, 2, 3, 4, 5, 6, 7, etc.-to-1, 2, 3, 4, 5, 6, 7, etc. for varying ratios and sensitivities of movement control. According to the varying sensitivities of control, and by way of example, a sonographer can change the ratio setting from, for example, a standard 1:1 stetting to, for example, a more sensitive 2:1 setting requiring twice the motion of the master sonography device 300 to move the slave sonography device 200. For example, a movement to the left of 10 cm on the master sonography device 300 in a 2:1 setting would result in a 5 cm movement to the left on the slave sonography device 200. This ability to change the sensitivity of the ratio between the systems can allow for improved performance with gross motions performed on the master sonography device 300 translating to finer/delicate motions at the slave sonography device 200, and vice versa.

In some aspects, the master sonography device 300 can further include a speaker 318 to receive audio communications from the operator and/or patient 204 at the slave sonography site 102. The master sonography device 300 can also include a microphone 320 so that the sonographer can verbally communicate with the operator and patient 204 at the slave sonography site 102. The master sonography device 300 also includes a display 322 so that the sonographer can see the patient 204, the transducer head 208 contacting the patient 204, or a combination thereof.

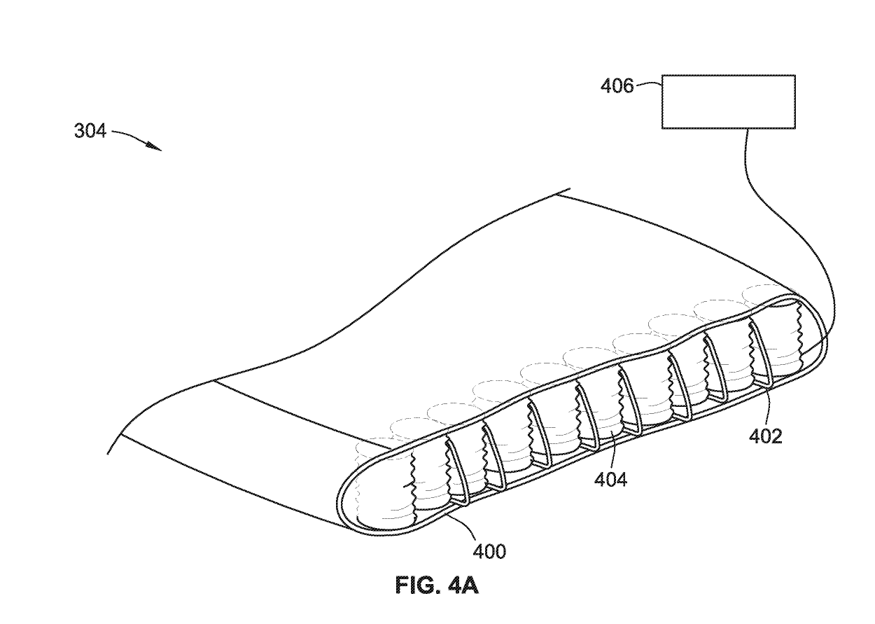

FIGS. 4A-4C show detailed views of the mock patient 304 upon which the sonography examination is performed at the master sonography site 104. The mock patient 304 is configured to be contoured from measurements acquired from the positioning system 210 at the slave sonography site 102. The measurements can be tailored for a particular examination type. Through the use of the arrangement data in the form of position measurements gathered from the positioning system 210, a spatially specific model of the patient 204 can be derived. The spatially specific model can be generated through various modeling techniques that translate a plurality of three-dimensional coordinates, vectors, planes, etc. into a three-dimensional model. Although described below with respect to a specific configuration, the mock patient 304 can manifest in a variety of different shapes, arrangements, configurations, etc., all of which allow for the shape and dimensions of the mock patient 304 to be modeled at the master sonography site 104 to allow for interaction by the sonographer on a proxy of the patient 204.

In some aspects, the mock patient 304 can include malleable inflatable fabrics with reinforcing support struts 402 that can be expanded or contracted to match the body shape of the patient 204 at the site of the sonography examination. The model derived from the slave sonography device 200 can be applied to the mock patient 304 by expanding or contracting the malleable inflatable fabrics to the desired body shape. Inflation can be performed using, but not limited to, a gas or a liquid. In some aspects, the malleable inflatable fabrics of the mock patient 304 can include components that can change in size to match the shape of the patient 204. For example, the components can include inflatable cylinders (as shown), spheres, squares, other polygons, etc. arranged within a fixed network and covered with a minimally flexible synthetic skin. Each sphere can be inflated to a specific size and/or shape and combined with the plurality of spheres to form the contour of the patient 204. Once formed, the fabric is pliable so as to offer little to no resistance to compression, allowing the transducer arm 306 of the master sonography device 300 to convey the pressure and compressibility characteristics of the tissue of the patient 204 to the sonographer. However, the outer layer of the fabric, such as the synthetic skin, is thick enough to prevent the sonographer from feeling the tactile sensation of the underlying inflated spheres and is of similar mechanical character so as to provide a similar friction interface between the transducer and synthetic skin allowing for an identical near-frictionless interface, as would be experienced by a sonographer during a bedside examination using gel. This can either manifest as a synthetic skin which is naturally near-frictionless or an analogue to normal human skin upon which gel can later be applied allowing for creation of the near-frictionless interface.

In some aspects, the mock patient 304 can be in communication (wired or wireless) with the master sonography device 300 to receive the model of the patient 204. In some aspects, the mock patient 304 can be in direct communication with the communications network 106 to receive the model of the patient 204, such as from the slave sonography device 200 or a third-party service provider (discussed below), independent from the master sonography device 300.

In some aspects, the mock patient 304 can be connected to the master sonography device 300, and the master sonography device 300 can control the mock patient 304 to conform to the margins of the patient 204 based on the received model. For example, the master sonography device 300 can control the manipulation of the inflatable spheres to configure the mock patient 304 in the shape of the patient 204 based on the received model, such as by controlling a pressurization system 406 connected to the mock patient 304 and valves connected to the various inflatable spheres. In some aspects, the mock patient 304 can be self-controlled to conform to the margins of the patient 204. For example, the mock patient 304 can include an internal or external pressurization system 406 connected to the inflatable spheres, and the mock patient 304 can be self-controlled to conform to the margins of the patient 204 based on the received model, independent of external control, such as external control from the master sonography device 300.

In some aspects, the master sonography device 300 can be guided by the physical parameters of the patient at the slave sonography device 200 without the necessity of a mock patient 304. One such embodiment allows the master sonography device 300 to respond to the physical environment of the slave sonography device 200 in real time without a physical proxy or mock patient 304 at the master sonography site 104. In such a configuration, when the transducer arm 306 is pulled down by the sonographer, the transducer arm 206 can respond in real time and mimic the same downward movement of the transducer arm 306 in a 1-to-1 motion. However, when the transducer arm 206 contacts upon a surface, e.g., a surface of the patient 204, the transducer arm 206 can transmit increased pressure/resistance experienced by the transducer arm 206 to the transducer arm 306 as a proportional resistance to motion. The proportional resistance to motion can provide the transducer arm 306 with the same resistance to motion as experienced by the transducer arm 206 on the patient. In other words, the system 100 can rely on force feedback at the master sonography device 300 to allow the transducer arm 306 to respond to the environment of the slave sonography device 200 as the transducer arm 206 without an object for a physical or tangible interface by the master sonography device 300.

In some aspects, the system 100 can be configured for augmented reality or virtual reality. The system 100 can be configured to allow the sonographer to visualize the slave sonography site 102 in real time and with a perspective as if the sonographer were located at the slave sonography site 102 while positioned at the master sonography site 104. The system 100 can include a video camera at the slave sonography site 102 that transmits real time video data (e.g., through standard definition video, high definition video at 1080p or 4K, stereovision/3D video, etc.) thereby allowing the sonographer another alternative way of perceiving the environment of the slave sonography site 102 and the patient 204's physical parameters to guide the examination.

In some aspects, the system 100 can be configured according to combinations of the foregoing, such as including the mock patient 304, force feedback between the slave sonography site 102 and the master sonography site 104, and configured according to an augmented reality or virtual reality. These systems can function independently or in concert with each other (or with other patient interface systems) to provide the a substantially seamless and substantially safe experience for both the sonographer and patient 204.

FIG. 4A shows the mock patient 304 in a deflated or substantially deflated state, such as before an examination. FIG. 4B shows the mock patient 304 in a first inflated state during an examination. By way of example, the mock patient 304 can be inflated according to FIG. 4B to mimic the abdomen of an average adult. FIG. 4C shows the mock patient 304 in a second inflated state during an examination. By way of example, the mock patient 304 can be inflated according to FIG. 4C to mimic the abdomen of an overweight adult. Thus, the mock patient 304 provides for the flexibility to mimic the margins of the patient 204 depending on the overall size, weight, etc. of the patient 204, in addition to the area of the examination on the patient 204.

Besides manipulation of a transducer arm and a transducer head 208, a sonographer typically performs other functions at a sonography device to perform a sonography examination. In the case of the slave sonography device 200 according to the present disclosure, these functions can be automated or initiated and controlled by the sonographer at the master sonography site 104, remote from the slave sonography device 200. Such other functions can include, for example, changing transducer heads and placing gel on the transducer head connected to the transducer arm prior to the examination. In accord with aspects of the present disclosure, these functions can be performed automatically by the slave sonography device 200 (e.g., without operator intervention at the slave sonography device 200) upon receipt of one or more commands from the sonographer at the master sonography device 300.

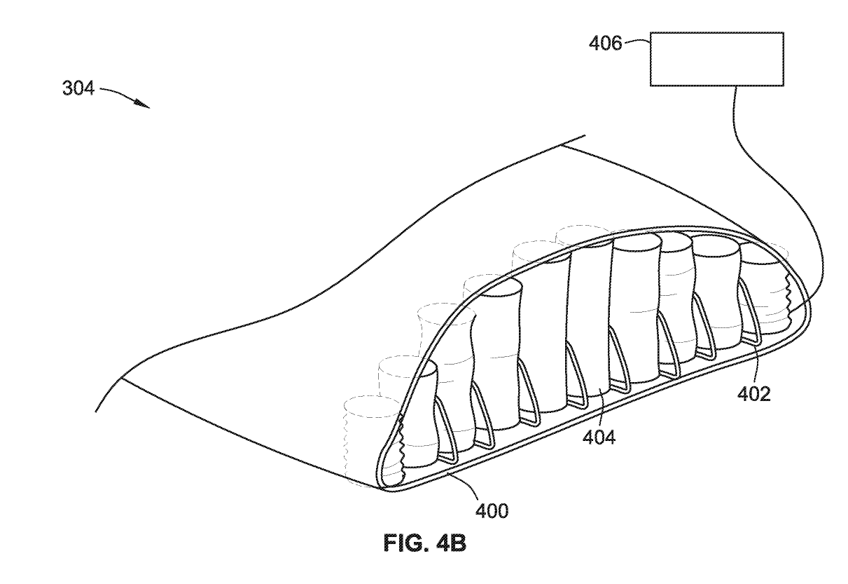

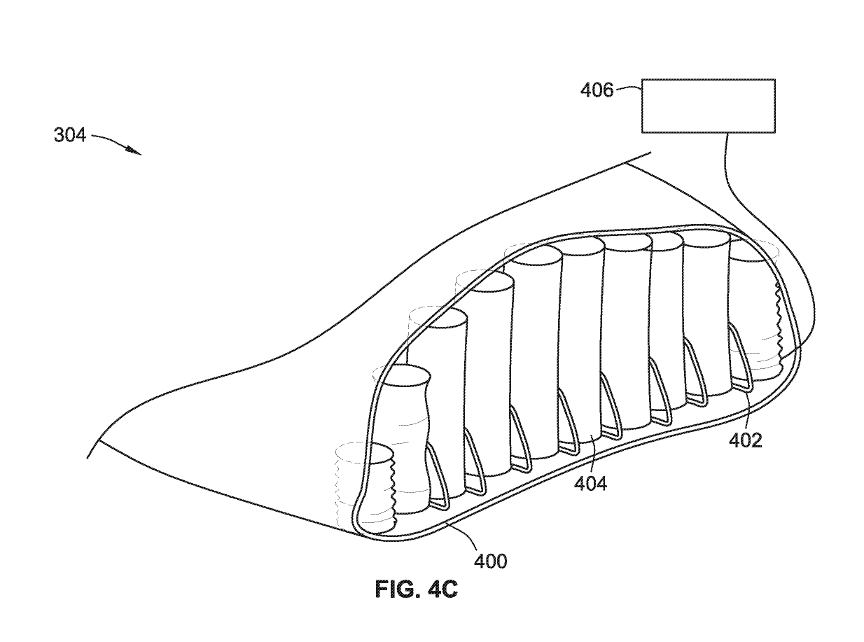

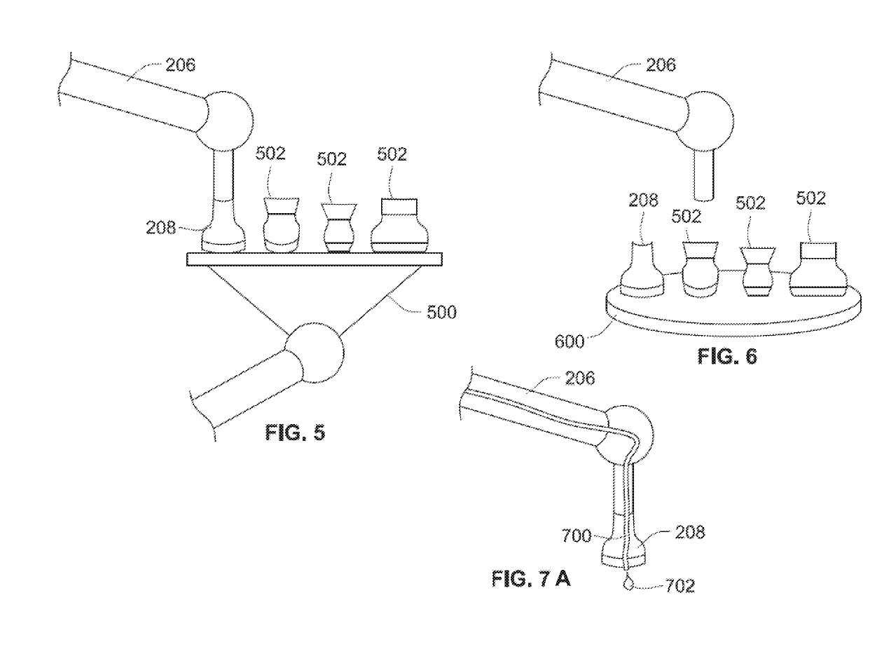

Referring to FIG. 5, in some aspects, the transducer arm 206 of the slave sonography device 200 can include several options for automatically changing the transducer head 208. According to one option, the slave sonography device 200 can include a loading/unloading arm 500 that can switch the transducer head 208 being used with one of a plurality of transducer heads 502 that are stored on the loading/unloading arm 500. Alternatively, another option includes the transducer heads being located on the base of the slave sonography device 200 (such as at a home position) and, when a switch needs to be made, the transducer arm 206 returns to the home position, unloads the current transducer head, moves over the new transducer head, and advances until the new transducer head (which is pointing with tail out) is loaded properly into the transducer arm 206.

Referring to FIG. 6, in some aspects, the transducer arm 206 can include a mechanism 600, such as a drum, that holds all of the transducer heads in a radial or a semi-radial fashion. When a specific transducer head is needed, the mechanism 600 rotates to the position of the specific transducer head and the transducer arm 206 of the slave sonography device 200 can perform a standard movement and operation for releasing the current transducer head 208 and attaching the desired transducer head 208. Similarly, the transducer arm 206 can store all of the transducer heads in this radial or semi radial fashion within the region of the transducer arm 206 on a rotatable wheel mechanism and simply rotate the desired transducer head into position as needed.

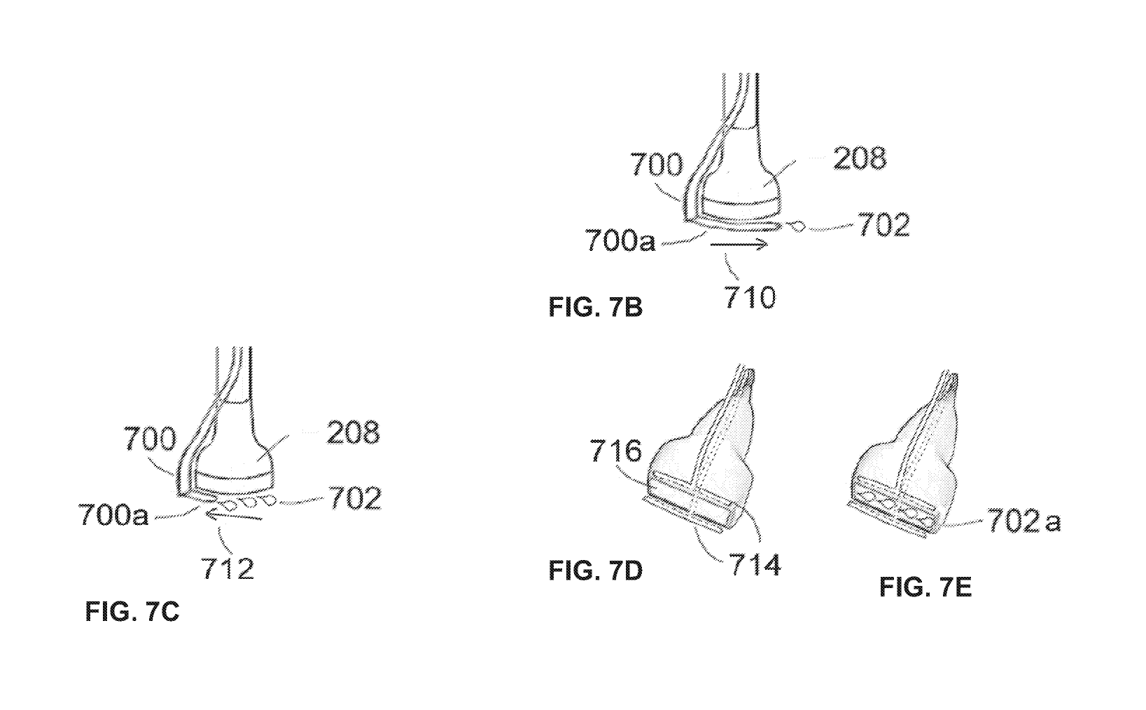

Referring to FIG. 7A, FIG. 7A shows a perspective view of a transducer head with a catheter for distributing gel on the transducer head, in accord with aspects of the present disclosure. With respect to an automatic gel dispenser, the slave sonography device 200 can include a catheter 700 that extends along the side of the transducer arm 206 and ends at the transducer head 208. Prior to an examination, the catheter 700 can extend across the surface of transducer head 208 and expel gel 702. Alternatively, prior to examination, the catheter 700 can dispense the gel 702 at the site of the patient 204 where the sonography examination will be performed. Further, control of the catheter 700 at any point during an examination can apply additional gel 702 to the transducer head 208 or the sonography site through the same techniques.

Referring to FIGS. 7B-7E, these figures show perspective views of alternative transducer heads with catheters for distributing gel on the transducer heads, in accord with aspects of the present disclosure. As shown in FIGS. 7B and 7C, the catheter 700 can include an extension 700a that can extend in a direction 710 across the transducer surface and expel gel 702 as it retracts back in a direction 712, thereby applying gel 702 across the entire length of the transducer surface. Alternatively, prior to examination, the catheter 700 can dispense the gel 702 at the site of the patient 204 where the sonography examination will be performed. Further, control of the catheter 700 at any point during an examination can apply additional gel 702 to the transducer head 208 or the sonography site through the same techniques.

Alternatively, referring to FIGS. 7D and 7E, the catheter 700 can bifurcate and flank the transducer head 208 along both its longitudinal surfaces 714 and can dispense gel 702 along both surfaces allowing gel 702 to be discharged upon the transducer surface 716 from both sides simultaneously and limiting interference with the transducer-patient interface. The catheter 700 can extend along the length of the transducer head on either side and can have multiple perforations allowing for gel 702 to be extruded along the length of the transducer head.

FIG. 8 illustrates a perspective view of the slave sonography device 200, in accord with aspects of the present disclosure. Although focusing on the slave sonography device 200, however, the illustration in FIG. 8 and the description below also apply to the master sonography device 300. As described above, the device 200 includes the transducer arm 206 and the transducer head 208. As illustrated in FIG. 8, the device 200 also includes a foundation 802. The foundation 802 provides a stable and secure platform for the device 200 to apply and/or maintain pressure against the patient. The foundation 802 can be a permanent attachment to the floor. Alternatively, the foundation 802 can be an ultrasound machine, scaffold mounts on the ceiling or wall, a portion of a gurney upon which the patient is lying, or the like. Alternatively, the foundation 802 can be a temporary attachment to a gurney or other portable object. The foundation 802 may be one of various different forms and configurations, such as, without limitation, a fixed floor mount, a mobile floor mount, a ceiling mount, a wall mount, or a furniture attachment, such as for a desk, a cabinet, a gurney/bed, or to a medical instrument machine, such as an ultrasound machine. The type of foundation 802 may depend on, for example, the location of the device 200, such as whether the room in which the device 200 is located is a dedicated examination or ultrasound room or a surgical suite with existing ceiling mounted scaffolding, etc.

The foundation 802 can be considered the first segment of the transducer arm 206, or the foundation 802 can be considered a separate element from the transducer arm 206. For example, the foundation 802 may include a base 808 and a stand 810 that extends up from the base 808. In such a configuration, the base 808 and the stand 810 may be immobile, such that the base 808 and the stand 810 cannot move relative to each other or relative to the patient. The base 808 and the stand 810 can be considered separate components of the device 200 from the arm 206. Alternatively, the foundation 802 may instead be an attachment point at the end of the arm 206, such as in the base of an arm positioning device that attaches directly to the gurney or to scaffold mounts. In which case, the foundation 802 can be considered part of the arm 206. In either case, the foundation 802 provides support for the arm 206 at a location that will not interfere with the patient (or the sonographer, in the case of a master sonography device 300).

The arm 206 can include plural segments and joints along its length from an initial segment or joint to a final joint connecting to the transducer head 208. As shown in FIG. 8, the device 200 can include segments 812a and 812b and joints 814a-814c. The joints 814a-814c can be various types of joints, such as hinge joints, ball joints, etc. The joints 814a-814c along the arm 206 can have one or more degrees of freedom or motion. By way of example, and without limitation, all of the joints 814a-814c, with the exception of the most distal joint 814c, can have a single or multiple degrees of freedom. Each degree of freedom of the joints 814a-814c may be the same degree of freedom or a different degree of freedom. For example, joints 814a and 814b may allow for a tilting motion. The tilting motion allows the segment attached to the joint to tilt along the axis of the joint as well as rotate substantially 360° within the plane defined by the joint both immediately proximal to the joint 814a-814c as well as immediately distal to the joints (these representing further degrees of motion). The rotational motion allows the segment attached to the joint to rotate orthogonally 3600 around the joint. Although disclosed and shown as including three joints 814a-814c and two segments 812a and 812b, the arm 206 can have more or fewer joints and/or segments. For example, a greater number of joints and segments can allow the arm 206 to conform to a greater number of positions, and a fewer number of joints may reduce manufacturing costs and complexity.

In the case of a master sonography device 300, the device 300 can be semi-passive. Specifically, the joints 814a-814c can provide active resistance to movement under the force of gravity to prevent the segments 812a and 812b from moving when not in use. In such a configuration, the arm 206 moves only in response to motion by the user. Specifically, the arm 206 can provide active assistance that is initiated and directed by the user moving the arm 206. Accordingly, a user can grasp the transducer head 208 or the distal-most segment 812b and move it to the desired location, and the device 200 will mechanically follow the intended path. In such a configuration, the joints 814a-814c provide immediate responsiveness to the user's active motions with little to no resistance and will hold their last positions when the user releases or halts movement.

Within the joints 814a-814c and/or the segments 812a and 812b can be one or more components (e.g., motors, servos, actuators, pumps, sensors, and the like) that sense and power movement of the arm 206. In the case of the slave sonography device 200, the components actively move the device 200 according to inputs received from the master sonography device 300 during a sonography procedure. In the case of a master sonography device 300, the components can aid movement of the device 300, such as to provide less strain to the operator during a procedure, and also generate the information that is transmitted to the slave sonography device 200. For example, servos and/or sensors within the joints can sense the direction, acceleration, and/or velocity of their motion. This information can then be delivered to a central processor associated with the device 300 and transmitted to other components within the device 300 to aid movement of the device 300 itself and/or to the slave sonography device 200. In the case of the master sonography device 300, the information is communicated back to those specific components determined to assist with motion in the required direction and with the required acceleration and velocity, thereby providing motion assistance. The information can be dynamically updated so that complex maneuvers can be communicated near-instantaneously to the various components to coordinate a fluid experience by the user so she will experience minimal to substantially no inertia from the mass of the arm.

In the case of the slave sonography device 200, the device 200 is entirely active based on controls received form the master sonography device 300. Thus, the components within the joints 814a-814c receive the direction, acceleration, and velocity of the corresponding joints 814a-814c of the master sonography device 300. This is dynamically updated so that complex maneuvers can be communicated near-instantaneously to the various servos and joints from the master sonography device 300 to coordinate a fluid experience by the patient.

The transducer head 208 extends from the most distal joint 814c on the arm 206. The transducer head 208 and the most distal joint 814c provide degrees of freedom of motion enabling 360° rotation as well as 360° tilting of an instrument connected to the transducer head 208. Similarly, the transducer head 208 provides pressure application assistance. For example, the transducer head 208 can include a linear coil actuator, or other mechanism that extends an attached instrument (not shown) linearly in a direction away from the transducer head 208. Accordingly, a user can position the transducer head 208 based on the rotational and tilting degrees of motion described above, and then apply pressure by activating the linear coil actuator within the transducer head 208. In one embodiment, the master sonography device 300 can determine movement parameters that describe the three-dimensional position, angulation, velocity, and pressure of the transducer head 308 as controlled by the sonographer. These movement parameters can then be transmitted to the slave sonography device 200 over the communications network 106 to control the slave sonography device 200 based on the control of the master sonography device 300 by the sonographer.

By allowing the sonographer to be remote from the patient 204, a sonography examination can be performed that is specific to the patient 204 by a sonographer who is an expert for that particular examination. A constant video and audio link between the sonographer and the patient 204 enable continuous communication to optimize patient 204 positioning and examination efficiency. Once completed, the information can be forwarded to the institutions radiology department or finalized by the tele-sonography affiliated radiology department or other relevant medical department.

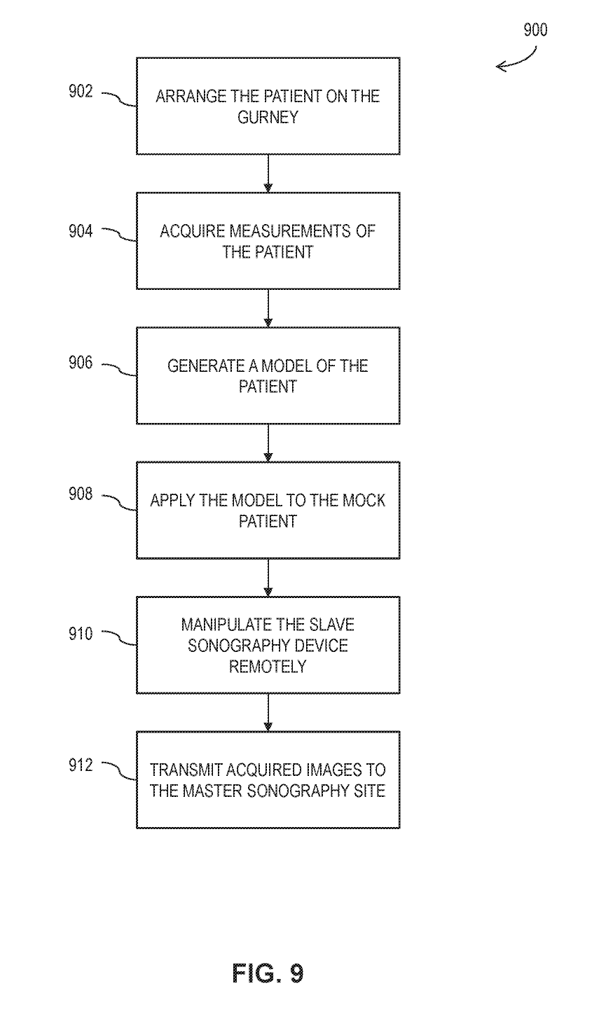

FIG. 9 is a flowchart of a procedure for real-time tele-sonography, in accord with aspects of the present disclosure. At step 902, the patient 204 is brought to a gurney 202 at the slave sonography site 102 by an operator of the slave sonography device 200, such as a nurse, a technician, an aid, a layperson, etc. The patient 204 is then arranged on the gurney 202 according an arrangement specific for the examination. For example, the patient 204 is arranged supine for imaging of the abdomen, prone or for imaging of the back, lateral decubitus for renal exams, etc.

The instructions for the arrangement of the patient 204 on the gurney 202 can be stored within the slave sonography device 200. The stored instructions can then be accessed by the operator, such as according to the specific examination that will be performed. Alternatively, the instructions can be transmitted to the slave sonography device 200 from the master sonography device 300. The transmitted instructions can subsequently be displayed on the display 222 of the slave sonography device 200, communicated audibly via the speaker 218 within at the slave sonography device 200, or a combination thereof.

After properly positioning the patient 204 on the gurney 202, and possibly confirming the operability of the system, the operator at the slave sonography site 102 can confirm that initiation has been completed, such as by pushing a confirmation button on the slave sonography device 200. In some aspects, the sonographer confirms the correct alignment of both the patient 204 and the transducer head 208 visually via the camera 216, which, as described above, can provide the sonographer's perspective and can be controlled to provide a specific view.

At step 904, the slave sonography device 200 acquires measurement parameters of the patient 204 to generate a model that is applied to the mock patient 304. The measurement parameters can be acquired by the positioning system 210 and can be any type of measurements that can translate the arrangement of the patient 204 to the arrangement of the mock patient 304 at the master sonography site 104.

At step 906, after acquiring the measurement parameters, a three-dimensional model of the patient 204 is generated. The model can be generated based on any modeling technique and according to any computer modeling format, application, and/or software. In some aspects, the model can be generated by the slave sonography device 200 and subsequently transmitted to the master sonography device 300. In some aspects, the model can be generated by the master sonography device 300 after the measurement parameters are transmitted to the master sonography device 300. In some aspects, the slave sonography device 200 can transmit the measurement parameters of the patient 204 to a third-party service provider also in communication with the communications network 106. The third-party service provider can then generate the model based on the measurement parameters and transmit the model to the master sonography device 300.

At step 908, after receiving the model at the master sonography device 300, the model is applied to the mock patient 304 to modify the margins of the mock patient 304 to conform to the margins of the patient 204. Application of the model to the mock patient 304 conforms the mock patient 304 to the shape of the patient 204 with respect to the site on the patient 204 upon which the sonography examination will be performed. Accordingly, despite the sonographer being remote from the patient 204, an accurate representation of the patient 204 is generated on which the sonographer can apply the transducer head 308 to perform the sonography examination. The sonographer benefits from a more realistic experience by having the tactile feedback of applying the transducer head 308 against the mock patient 304, further enhanced by feedback information from the slave sonography device 200 regarding pressure and resistance being transmitted to the patient 204 during the examination.

Once modification of the configuration of the mock patient 304 is complete, the sonographer at the master sonography device 300 can begin the examination by placing the transducer head 308 on the mock patient 304.

At step 910, the sonographer manipulates the transducer head 308 against the mock patient 304 to cause the transducer head 208 at the patient to move relative to the patient 204. The transducer head 308 of the master sonography device 300 sends real-time commands to the slave sonography device 200, which already has the transducer head 208 of the slave sonography device 200 on the patient 204, for acquiring sonography images of the patient 204. When the sonographer moves the transducer arm and the transducer head 308, the master sonography device 300 transmits every motion and vector of acceleration to the slave sonography device 200 allowing for a one-to-one modeling of the master system onto the slave system. Calibration of the location of the transducer heads 208 and 308 at the start of the examination can be performed by the system 100 by actively moving the transducer head 308 to the corresponding spatial location of the transducer head 208, or vice versa. Alternatively, a passive/semi-passive system 100 can require the transducer head 308 to be moved by the sonographer to a start location or to a pre-set position to coordinate the two transducer heads. After location of both transducer heads 208 and 308 is calibrated, the system 100 can allow the examination to begin.

In some aspects, the slave sonography device 200 can relay information back to the master sonography device 300. The information can subsequently be displayed in real time to provide a more realistic experience for the sonographer at the master sonography device 300. More specifically, during the examination, the slave sonography device 200 actively transmits physical data back to the master sonography device 300. The physical data can include, for example, pressure data indicating an amount of pressure being applied by the transducer arm of the slave sonography device 200 to the patient 204. The pressure data can be used by the master sonography device 300 to resist the sonographer's efforts proportional to the resistance experienced by the slave transducer due to the patient 204's reactive physical resistance, or transmit that resistance through active modification to the pressure of the relevant locations in the mock patient to provide the sonographer with real-time tactile information to help guide the sonographer as if the sonographer were at the patient 204.

At step 912, the images obtained from the patient 204 are then transmitted back to the master sonography device 300 so that the sonographer can perform the examination as if the sonographer were at the patient 204. The images can be displayed in real time on the display 322 of the master sonography device 300 so that the sonographer can alter the sonography examination based on the quality of the images being generated. Further, the images can be stored at the master sonography device 300 or another storage medium for later retrieval by, for example, a radiologist for examination of the images.

Voice communication from the sonographer to the patient 204 is possible at all times. If the patient 204 needs to adjust to a new position, the master sonography device 300 can be recalibrated to reorient to the new orientation of the patient 204 based on information from the positioning system 210. Upon completion of the examination, which can be indicated by, for example, depression of a button (e.g., manual or graphical user interface) on the master sonography device 300, all components of the slave sonography device 200 can retract away from the patient 204 to provide a safe and unencumbered exit from the gurney 202 or examination table.

The technology discussed herein is specifically designed to accomplish the goals of removing the need for a sonographer present at a patient's bedside and allowing a remote radiologist or other health care professional to examine high-quality images with unparalleled precision and convenience. The technology discussed herein can be used in primary care and family medicine offices, subspecialty clinics, and private practices in the rural and urban community centers alike. Similarly, hospitals in more secluded regions, and rural and some suburban secondary care centers are specifically positioned to greatly benefit from the technology discussed herein. Similarly, this technology can be installed upon ambulances and mobile care stations or portable care units to be used as needed on site during disasters, military maneuvers or while transporting patients to higher levels of care. All of these institutions share the common need of reliable sonography examinations but lack access to inexpensive resources, experienced sonographers, and imaging interpretation. Their patients would alternatively have to pursue third-party services, prolonging the time before a diagnosis can be made or proper care provided, increasing time and expense and removing potential billable income from the referring institution. With a single purchase, institutions will be able to provide expert sonography care for their patient population regardless of indication. The technology discussed herein provides a variety of benefits beyond the obvious reduced cost of obviating the need for a sonographer at the patient's bedside. It allows for super-specialization as sonographers can focus on only a single procedure or a specific group of examinations and ensures that the patient receives an examination only from the most qualified sonographer super-specialized for that examination. Furthermore, the technology discussed herein allows for real-time evaluation by the radiologist if necessary to provide insight to improve the diagnostic yield of the study as it is being performed. The technology discussed herein can provide the most advanced sonography services to any facility regardless of its location. Moreover, facilities pay only for service provided and have only the initial device purchase expense.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. A tele-sonography system comprising:
a master sonography device including a master transducer head, the master sonography device being configured to determine movement parameters describing movement of the master transducer head by a sonographer at the master sonography device;
a slave sonography device, remote from the master sonography device, including a slave transducer head and a positioning system, the slave sonography device being configured to move the slave transducer head based on the movement parameters received from the master sonography device, and the positioning system being configured to generate patient parameters describing margins of a patient upon which a sonography examination is performed at the slave sonography device; and
a mock patient, at the master sonography device, configured to conform to the margins of the patient based on the patient parameters and mimic the patient at the master sonography device during the sonography examination,
wherein manipulation of the master transducer head applied against the mock patient controls movement of the slave transducer head applied against the patient.

2. The system of claim 1, wherein the mock patient includes malleable fabrics surrounding support struts.

3. The system of claim 2, wherein the mock patient includes components configured to change in size to cause the mock patient to conform to the margins of the patient.

4. The system of claim 3, wherein the components include a plurality of inflatable spheres.

5. The system of claim 1, wherein the positioning system includes a plurality of tactile sensors placed about the patient at an area of the examination, and the plurality of tactile sensors are configured to generate the patient parameters.

6. The system of claim 1, wherein the positioning system includes a laser imaging system and one or more detectors, and the laser imaging system and the one or more detectors are configured to generate the patient parameters.

7. The system of claim 1, wherein the slave sonography device includes a catheter configured to apply gel on the slave transducer head based on one or more commands received from the master sonography device.

8. The system of claim 1, wherein the slave sonography device is configured to exchange the slave transducer head with a selected one of a plurality of additional slave transducer heads based on one or more commands received from the master sonography device.

9. The system of claim 1, wherein the master sonography device and the mock patient are at a master sonography site, the slave sonography device and the patient are at a slave sonography site, remote from the master sonography site, and information is exchanged between the master sonography site and the slave sonography site for control of the slave transducer head based on the master transducer head over a communications network.

10. The system of claim 1, wherein the master sonography device includes a master transducer arm from which the master transducer head extends, and the master sonography device is configured to determine the movement parameters describing movement of the master transducer arm and the master transducer head by the sonographer.

11. The system of claim 1, wherein the slave sonography device includes a slave transducer arm from which the slave transducer head extends, and the slave sonography device is configured to move the slave transducer arm and the slave transducer head based on the movement parameters received from the master sonography device.

12. A method of performing tele-sonography comprising:
positioning a patient relative to a slave sonography device, the slave sonography device including a slave transducer arm, a slave transducer head, and a positioning system, the slave sonography device being configured to move the slave transducer head based on movement parameters received over a communications network;
acquiring patient parameters describing margins of the patient relative to the slave sonography device;
generating a model of the patient based on the patient parameters;
applying the model to a mock patient, adjacent a master sonography device and remote from the slave sonography device, to conform margins of the mock patient to the margins of the patient; and
acquiring sonography images at the slave sonography device based on control of the slave sonography device by the master sonography device.

13. The method of claim 12, wherein the master sonography device includes a master transducer arm and a master transducer head, and manipulation of the master transducer arm and the master transducer head against the mock patient by a sonographer controls manipulation of the slave transducer arm and the slave transducer head against the patient for acquiring the sonography images.

14. The method of claim 12, wherein the positioning system generates the model based on the patient parameters.

15. The method of claim 12, wherein the mock patient includes malleable fabrics surrounding support struts.

16. The method of claim 15, wherein the mock patient includes components configured to change in size to cause the mock patient to conform to the margins of the patient.

17. The method of claim 16, wherein the components include a plurality of inflatable spheres, polygons, or a combination thereof.
